(19) United States
(12) Patent Application Publication
(10) Pub. No.: US 2026/0294993 A1
(43) Pub. Date: Oct. 1, 2026

(54) DIETARY SUPPLEMENT FOR MUSCLE AND JOINT PAIN

(71) Applicant: KENVUE BRANDS LLC, Summit, NJ (US)

(72) Inventors: Eleanor F. Small, Philadelphia, PA (US); Cameron K. Mylroie, St. Michael, MN (US); Shaness A. Grenald, East Orange, NJ (US); Michael L. Costante, Chalfont, PA (US); Bruce P. Smith, Blue Bell, PA (US); Jennifer Cooper, Provo, UT (US)

(21) Appl. No.: 19/096,504

(22) Filed: Mar. 31, 2025

(51) Int. Cl. A61K 36/48; A61K 9/20; A61K 36/42; A61K 36/9066

(52) U.S. Cl. CPC A61K 36/48; A61K 9/2009; A61K 9/2054; A61K 36/42; A61K 36/9066

(57) ABSTRACT

Drug-free, dietary supplement compositions to prevent or alleviate pain in mammals, in particular muscle and joint pain, joint discomfort, and/or joint stiffness are provided. The compositions can include a combination of Tamarindus indicia seed extract, Curcuma longa rhizome extract, and Cucumis sativus algae extract. The compositions can be in the form of a compressed tablet.

### FIELD

The present invention relates to drug-free, dietary supplement compositions to prevent or alleviate pain, in particular muscle and joint pain, support and/or improve joint comfort, and increase range of joint motion in mammals. The composition may include a combination of Tamarindus indicia seed extract, Curcuma longa rhizome extract, and Cucumis sativus algae extract. The composition may be a compressed tablet.

### BACKGROUND

Pain is a common experience that may be of short duration (called “acute pain”) or of longer duration, for example, six months or longer (called “chronic pain”). Pain may include, for example, muscle and joint pain of the body. As used herein, “pain relief” refers to the alleviation of pain at one or more locations of the body. In addition to pain, joint discomfort and a decreased range of joint motion are conditions that may negatively impact a person's quality of life and treatment of the same may be costly. Treatment options for pain, joint discomfort, and joint stiffness that are effective and inexpensive are desirable.

Over-the-counter (“OTC”) and prescription (“Rx”) drugs may be prescribed or used to relieve pain, joint discomfort, and/or joint stiffness. However, while OTC and prescription drugs are highly effective, many consumers desire pain relief or other relief from joint discomfort and/or stiffness through drug-free or natural products. Such products may be dietary supplements that provide health benefits when ingested by a consumer. Such health benefits may include pain relief, for example, muscle and joint pain relief, and/or may include support and/or improved joint comfort and an increased range of joint motion. Examples of a dietary supplement for pain relief or other relief from joint discomfort and/or stiffness are discussed below.

Additionally, while there are many dietary supplements on the market, many of them are in a format that is undesirable to consumers. Similar to pharmaceutical dosage forms, dietary supplements may be prepared in a capsule or tablet form, but often these capsules and tablets are large in size and make swallowing difficult for consumers. Reducing the size of the dosage form for dietary supplements is complex. First, because many dietary supplement ingredients degrade over time, dietary supplement products often include overages of such dietary supplement ingredients to ensure that they do not degrade below the effective amount required for a claimed health benefit. These overages often increase the size of the dosage form. Second, many dietary supplement ingredients are difficult to process into tablet form, for example requiring large amounts of excipients to create a stable dosage form. Larger amounts of excipients will again increase the size of the dosage form. For this reason, capsules are more predominant than tablets in dietary supplement products. However, capsules are generally less preferred by consumers because they are more difficult to swallow. These problems both contribute to the large physical size of dietary supplements on the market.

Therefore, an additional need exists to provide dietary supplements in a smaller dosage form to enhance the ease of swallowing. Examples of a dietary supplement in a compressed tablet form are discussed below.

### SUMMARY

The present invention provides compositions to alleviate pain. Such compositions can include a combination of Tamarindus indicia, Curcuma longa and Cucumis sativus. In certain embodiments, the Tamarindus indicia can be Tamarindus indicia seed extract. In certain embodiments, the Curcuma longa can be Curcuma longa rhizome extract. In certain embodiments, the Tamarindus indicia seed extract and the Curcuma longa rhizome extract can be used in a ratio ranging from about 1:3 to about 3:1, or about 2:1.

In certain embodiments, the Cucumis sativus can be in an amount of about 20 mg, or about 24 mg. In certain embodiments, the Tamarindus indicia can be in an amount of about 157.5 mg, the Curcuma longa can be in an amount of about 78.75 mg and the Cucumis sativus can be in an amount of about 24 mg.

In certain embodiments, the Tamarindus indicia, Curcuma longa and Cucumis sativus can be in a ratio of about 150:75:20.

In certain embodiments, the composition can be a pain supplement in the form of a tablet, caplet or hard-shell capsule. In particular embodiments, the composition can be in the form of a compressed tablet.

In certain embodiments, the composition can further include a compression aid. In particular embodiments, the compression aid can consist of a combination of microcrystalline cellulose and tricalcium citrate. The microcrystalline cellulose can be in an amount of about 50 mg and the tricalcium citrate can be in an amount of about 258.77 mg.

In certain embodiments, the composition can have a hardness of at least 10 kP, or from about 11.2 kP to about 12.5 kP.

In certain embodiments, the composition can be free of any active pharmaceutical ingredient (API). In certain embodiments, the composition can be free of dyes. In certain embodiments, the composition can be vegetarian, vegan and/or gluten-free.

### DETAILED DESCRIPTION

As used herein, the term “about” refers to within 5% weight, within 4% weight, within 3% weight, within 2.5% weight, within 2% weight, or within 1% weight of a disclosed value.

As used herein, “mg” refers to milligram. And, the term “w/w” refers to weight by weight, or the proportion of a particular substance within a mixture, as measured by weight or mass. All percentages refer to percentage by weight, unless indicated otherwise.

As used herein, the term “dosage form” applies to any solid composition designed to contain a specific pre-determined amount (dose) of a certain ingredient, for example an active ingredient as defined below. Suitable dosage forms may be similar to those used in pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. In a particularly preferred embodiment, the dosage form is an orally administered system for delivering an active ingredient to the gastro-intestinal tract of a human.

The dosage form of the present invention may be referred to as a “Dietary Supplement” and preferably contains one or more dietary supplement ingredients. Dietary supplement ingredients broadly include, for example, minerals, vitamins, herbal or natural products and other ingestible preparations added to the diet to benefit health. Dietary supplement ingredients may also be described herein as “active ingredients.” Dietary supplement ingredients, however, do not include active pharmaceutical ingredients as defined herein.

### Examples of Dietary Supplement Ingredients:

In one embodiment, a composition to alleviate pain may include a combination of dietary supplement ingredients Tamarindus indicia and Curcuma longa. The Tamarindus indicia may be Tamarindus indicia seed extract. The Curcuma longa may be Curcuma longa rhizome extract. The Tamarindus indicia seed extract and the Curcuma longa rhizome extract may be used in a ratio ranging from 1:3 to 3:1.

Tamarindus indicia, also known as tamarind, is a hardwood tree that grows in Africa, India, Pakistan and other tropical regions. The tamarind tree produces bean-like pods filled with seeds surrounded by a fibrous pulp. Historically and under traditional medicine, tamarind has been used to treat diarrhea, constipation, fever and malaria, and has also been used to promote wound healing. The polyphenols in tamarind have antioxidant and anti-inflammatory properties that have been used to protect against heart disease, cancer and diabetes.

Curcuma longa, also known as tumeric, is a flowering plant in the ginger family native to India and Southeast Asia. Tumeric has been used to treat numerous medical ailments, including, but not limited to, liver obstruction, jaundice, ulcers, inflammation, cough, cold, dental issues, indigestion, skin infections, and wounds.

It has been shown that the combination of Tamarindus indicia and Curcuma longa, for example under the tradename Tamaflex® from Laila Nutraceuticals, demonstrates additional health benefits. For example, the combination of Tamarindus indicia and Curcuma longa may reduce joint discomfort, and increase joint and muscle function and flexibility in as little as five days. The combination of Tamarindus indicia and Curcuma longa may deliver fast-acting, long-lasting and protective benefits against joint and muscle pain. The combination of Tamarindus indicia and Curcuma longa may help improve functional performance in climbing up and down stairs, and may support an increased range of joint motion.

The product under the tradename Tamaflex® from Laila Nutraceuticals is a synergistic combination of therapeutically-efficacious amounts of Tamarindus indica seed extract and Curcuma longa rhizome extract for applications including inflammation, joint function, and collagen breakdown. The Tamaflex® product is patented under U.S. Pat. No. 11,147,850 (the '850 patent), which is incorporated herein by reference. In particular, the '850 patent discloses the use of Tamarindus indica seed extract and Curcuma longa rhizome extract in a ratio ranging from 1:3 to 3:1, for example 2:1.

The Tamaflex® product is known to contain six parts (w/w) Tamarindus indicia seed extract, three parts (w/w) Curcuma longa rhizome extract and one part (w/w) excipients. The excipient portion of the Tamaflex® product is a combination of 80% (w/w) microcrystalline cellulose powder and 20% (w/w) Syloid silica. This information is referenced in the article titled “A Combination of Tamarindus indica seeds and Curcuma longa Rhizome Extract Improves Knee Joint Function and Alleviates Pain in Non-Arthritic Adults Following Physical Activity,” Rao et al., International Journal of Medical Sciences, 2019; 16 (6): 845-853, which is incorporated herein by reference.

In an example, the pain supplement may incorporate the Tamaflex® product at about 250 mg. In this amount of Tamaflex® product, the Tamarindus indicia seed extract is about 150 mg and the Curcuma longa rhizome extract is about 75 mg.

In an example, the pain supplement may incorporate the Tamaflex® product at about 262.5 mg. In this amount of Tamaflex® product, the Tamarindus indicia seed extract is about 157.5 mg and the Curcuma longa rhizome extract is about 78.75 mg.

In an example, the Tamarindus indicia and/or Curcuma longa may be in an amount of about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 255 mg, about 260 mg, about 265 mg, about 270 mg, about 275 mg, or preferably about 262.5 mg.

In an example, the pain supplement may include an extract or standardized extract of cucumber, also known as Cucumis sativus in an amount of about 20 mg, or about 24 mg. In one example, the product sold under tradename Q-Actin® from IminoTech, Inc. may be used. Q-Actin® is an extract sourced from select cucumbers (Cucumis sativus L.) with high levels of ido-BR1 that soothes joint discomfort and increases flexibility and function. U.S. Pat. No. 9,326,977 may cover this proprietary extract, or similar Cucumis sativus extracts and is incorporated by reference herein.

In an example, the Tamarindus indicia and/or Curcuma longa may be in an amount of about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 255 mg, about 260 mg, about 265 mg, about 270 mg, about 275 mg, or preferably about 262.5 mg. Cucumis sativus may be in an amount of about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, or preferably about 24 mg.

In an example, the Tamarindus indicia is in an amount of about 150 mg, the Curcuma longa is in an amount of about 75 mg and the Cucumis sativus is in an amount of about 20 mg. In a further example, the Tamarindus indicia, Curcuma longa and Cucumis sativus are in a ratio of about 150:75:20, or about 1:2:7.5.

In an example, the Tamarindus indicia is in an amount of about 157.5 mg, the Curcuma longa is in an amount of about 78.75 mg and the Cucumis sativus is in an amount of about 24 mg. In a further example, the Tamarindus indicia, Curcuma longa and Cucumis sativus are in a ratio of about 1:2:6.6.

### Examples of Dosage Format:

According to an example, the preceding pain supplement compositions may be in a form of a tablet or caplet. As used herein the term “tablet” refers to a solid form prepared by compaction of powders on a tablet press, as well known in the pharmaceutical arts. Tablets can be made in a variety of shapes, including round, or elongated, such as flattened ovoid or cylindrical shapes. A “caplet” is defined as a tablet covered by a film coating.

According to another example, the pain supplement compositions may be in the form of a capsule or hard-shell capsule. As used herein the term “hard-shell capsule” refers to a solid form prepared by the filling of powder into a shell which is closed and sealed. The powder may be comprised of the pain supplement ingredients and/or excipients that are used for a tablet core. The shell may be comprised of gelatin or materials from non-animal sources such as polymers, vegetable-based films or starch.

According to an example, the capsule may include an additional band. The band may be comprised of gelatin or a film-based polymer and cover the seam of the capsule, providing an additional tamper evident feature. Other forms, such as soft gels, sachets, powders, gummies and chewable tablets may also include a tamper evident feature.

According to an example, the pain supplement compositions may be in the form of a compressed tablet. A “compressed tablet” is defined as solid dosage form comprised of a powder or powder blend compressed with sufficient force to allow for independent handling. As a comparison, the force used on the powder material filled within a hard-shell capsule is referred to as “tamping” of the powder. However, such tamping of the powder alone does not provide sufficient force for independent handling. The powder must be encapsulated and handled within the hard-shell capsule portion. This contrasts with the compressed tablet which may be handled independently.

The core (or substrate) or the tablet, caplet or capsule may be any solid form. The core may be prepared by any suitable method, for example, the core may be a compressed dosage form, or may be molded. As used herein, “substrate” refers to a surface or underlying support, upon which another substance resides or acts, and “core” refers to a material that is at least partially enveloped or surrounded by another material. For the purposes of the present invention, the terms may be used interchangeably: i.e., the term “core” may also be used to refer to a “substrate.” Preferably, the core comprises a solid, for example, the core may be a compressed or molded tablet or caplet. Alternatively, the core may be a suppository, or a confectionery form such as a lozenge, nougat, caramel, gummy, chewable tablet, fondant, capsule fill or fat-based composition.

The “core weight” of the tablet, caplet or capsule may be about 1,000 mg or less, or about 950 mg or less, or about 900 mg or less, or about 850 mg or less, or about 800 mg or less, or about 750 mg or less, or about 700 mg or less, or about 650 mg or less, or about 600 mg or less, or about 550 mg or less, or about 500 mg or less, or about 450 mg or less, or about 400 mg or less, or about 350 mg or less, or about 325 mg or less. According to one example, the core weight of the tablet, caplet or capsule may be about 650 mg.

The core may be pressed of a blend of suitable dietary supplement ingredients and excipients which may be either their natural color, including white, or can be conventionally colored as desired to provide a core of any desired color.

According to an embodiment, the core may contain a disintegrant and/or a superdisintegrant. Suitable disintegrants for making the core, or a portion thereof, by compression, include, e.g., sodium starch glycolate, croscarmellose sodium, cross-linked polyvinylpyrrolidone, cross-linked starches, microcrystalline cellulose, and the like. According to an example, the superdisintegrant may be present as a percentage of the weight of the core from about 0.05 percent to about 10 percent.

According to an example, the core may comprise a compressible filler, also referred to as a filler, tamping aid or compression aid. Compression aids facilitate the compression step used in forming compressible tablets dosage forms whereas tamping aids facilitate the tamping step of the capsule fill process. Compression aids include but are not limited to microcrystalline cellulose, directly compressible microcrystalline cellulose, tricalcium citrate, dicalcium phosphate, tricalcium phosphate, celluloses, water insoluble celluloses, dextrose, starch, cornstarch and modified starches. Suitable fillers include but are not limited to starch and modified starches. According to an example the core may comprise more than one compressible fillers, including a combination of microcrystalline cellulose and tricalcium citrate. In certain embodiments, the core can include a combination of tricalcium citrate and microcrystalline cellulose in a ratio of about 5.18:1.

In an example, the dietary supplement ingredients (e.g., Tamarindus indicia, Curcuma longa and Cucumis sativus) and the compression aid (e.g., tricalcium citrate) are in a ratio of about 1.11:1. In an example, the Tamaflex® product and the compression aid (e.g., tricalcium citrate) are in a ratio of about 1.01:1.

In an example, the dietary supplement ingredients (e.g., Tamarindus indicia, Curcuma longa and Cucumis sativus), tricalcium citrate and microcrystalline cellulose are in a ratio of about 5.73:5.18:1. In an example, the Tamaflex® product, tricalcium citrate and microcrystalline cellulose are in a ratio of about 5.25:5.18:1.

According to an example, the core may include a further compression aid, for example Hydroxypropylcellulose, commercially available under the tradename Klucel™ EXF Ultra HPC from the Ashland Corporation.

According to an example, the core may include a lubricant, for example magnesium stearate or stearic acid. A “lubricant” is defined as a material which aids in the flow of a powder or powder blend, allowing for processibility of solid dosage forms, including blending and compression.

In an example, the dietary supplement ingredients may be subject to raw material variability or degradation over time. As such, the dietary supplement ingredients may be included in the dosage form at about 1%, about 2%, about 3%, about 4%, about 5%, about 6%, about 7%, about 8%, about 9%, about 10%, about 12%, about 15%, about 18% or about 20% higher than the intended dietary supplement ingredient dosage amount, also known as “overages.” For example, a pain supplement with an intended 150 mg of a dietary supplement ingredient may include that dietary supplement ingredient at about 5% to 10% higher amount to ensure that least 150 mg of the dietary supplement ingredient is present in the dosage form when consumed.

The dietary supplement ingredient or excipient ingredients may be present in the dosage form in any form. For example, one or more dietary supplement ingredients may be dispersed at the molecular level, e.g., melted or dissolved, within the dosage form, or may be in the form of particles, which in turn may be coated or uncoated. If a dietary supplement ingredient is in the form of particles, the particles (whether coated or uncoated) typically have an average particle size of about 1-2000 microns. In another preferred embodiment, the particles are granules or pellets having an average particle size of about 10-500 microns, preferably about 20-500 microns, most preferably about 20-400 microns.

According to an example, disintegration of the tablet without agitation can be observed at less than about 25 minutes, e.g., less than about 20 minutes, e.g., less than about 15 minutes, e.g., less than about 10 minutes, e.g., less than about 5 minutes, e.g., less than about 60 seconds, e.g., less than about 30 seconds, e.g., less than about 15 seconds, e.g., less than about 10 seconds, or e.g., less than about 5 seconds.

In certain examples, the core may be covered with a coating. Any composition suitable for film-coating a tablet may be used as a coating according to the present invention. Suitable compositions for use as coatings include those manufactured by Colorcon, a division of Berwind Pharmaceutical Services, Inc., 415 Moyer Blvd., West Point, PA 19486 under the tradename “OPADRY®” (a dry concentrate comprising film forming polymer and optionally plasticizer, colorant, and other useful excipients). Additional suitable coatings include one or more of the following ingredients: cellulose ethers such as hydroxypropylmethylcellulose (HPMC), hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, opacifiers.

Coatings may include water soluble polymers selected from the group consisting of hydroxypropylmethyl cellulose, hydroxypropyl cellulose, methyl cellulose, polymethacrylates, polyvinyl alcohol, polyvinyl alcohol: polyethylene glycol copolymers and mixtures thereof.

According to an example, the average thickness of the coating is preferably in the range from about 1 micron to about 150 microns, or from about 50 microns to about 90 microns, or from about 10 microns to about 90 microns, or from about 20 microns to about 80 microns, or from about 30 microns to about 70 microns.

In one example, the coating may be from about 10 percent to about 50 percent, e.g., from about 15 percent to about 20 percent of HPMC. The dried coating may be present in an amount, based upon the dry weight of the core, from above about 0 percent to about 5 percent, or from about 1 percent to about 4 percent, or from about 2 percent to about 3 percent, or from about 1 to about 2 percent. The coating composition may be optionally tinted or colored with colorants such as pigments, dyes and mixtures thereof.

Alternatively, the dosage form, or pain supplement, may be free of dyes. As used herein, the term “free of” denotes the complete absence of an ingredient. Dyes have been traditionally added to dosage forms for elegance and to mask discoloration. However, it has been found that some patients develop or have allergies to or are sensitive to dyes. Therefore, dye-free dosage forms may be desirable.

The “total coated weight” of the tablet, caplet or capsule may be about 1,000 mg or less, or about 950 mg or less, or about 900 mg or less, or about 850 mg or less, or about 800 mg or less, or about 750 mg or less, or about 700 mg or less, or about 650 mg or less, or about 600 mg or less, or about 550 mg or less, or about 500 mg or less, or about 450 mg or less, or about 400 mg or less, or about 350 mg or less, or about 325 mg or less. According to one example, the total coated weight of the tablet, caplet or capsule may be about 658.13 mg. According to one example, the “core weight” of the tablet, caplet or capsule may be about 650 mg.

In certain examples, the pain supplement compositions may be in a form of a compressed tablet. Compressed tablets are produced through the addition of a compression force to a powder or powder blend, where the resulting tablet can be transported or handled independently without breakage. Tablets are often compressed on rotary compression machines and using compression forces and speed which adds stress and/or heat to the powder blend. This stress can affect the ingredients in the powder blend, and for some ingredients, in particular active ingredients, can lead to tablet failures. These failures can be present as tablet breakage (friability), insufficient hardness, unacceptable disintegration, or sticking and picking on the face of the tablet punches. Sticking and picking can also lead to loss of portions of tablets on tooling surfaces and uneven tablet weight variation. The issues of sticking and picking can be compounded for tablets that are debossed or embossed as material can adhere to these surfaces upon compression. Pain supplement compositions of the present disclosure in compressed tablet form advantageously achieved a desired hardness without sticking and picking on machine tooling, indicating that such formulations have reproducible compression characteristics and can be utilized to provide dietary supplement ingredients in tablet form at scale.

In one example, the dosage form may be free of drugs or any active pharmaceutical ingredient (API). A “drug” is defined as a substance recognized by an official pharmacopoeia or formulary; and/or a substance intended for use in the diagnosis, cure, mitigation, treatment, or prevention of disease; and/or a substance (other than food) intended to affect the structure or any function of the body; and/or a substance intended for use as a component of a medicine but not a device or a component, part or accessory of a device. Biological products are included within the definition of “drug” and are generally covered by the same laws and regulations, but differences exist regarding their manufacturing processes (chemical process versus biological process). An “active pharmaceutical ingredient” or “API” is defined any substance that is intended for incorporation into a finished drug product and is intended to furnish pharmacological activity or other direct effect in the diagnosis, cure, mitigation, treatment, or prevention of disease, or to affect the structure or any function of the body.

In an example, the dosage form, or pain supplement, may be vegetarian, vegan and/or gluten-free, as defined by industry standards and/or as certified by an independent lab.

In an example, the dosage form, or pain supplement, may deliver enhanced pain relief to customers. For example, the dosage form, or pain supplement, may deliver one or more of the following: 1) improved muscle and joint comfort in just seven (7) days; 2) supports a healthy inflammation response for occasional overworked muscles and stressed joints; 3) enhances occasional stress joints occurring as part of the aging process; 4) reduces aches and pains; 5) works three times faster than glucosamine and chondroitin; 6) reduces muscle soreness; and/or 7) wakes up tired muscles and joints.

In an example, the dosage form, or pain supplement may provide additional benefits to the consumer, for example a smaller size, a smaller dose, or ease of swallowability.

### EXAMPLES

The following examples set forth below further illustrate embodiments of the present invention. All amounts are given in weight percent. The present invention is not limited to the examples contained therein.

### Example 1

TABLE 1
Active Ingredients for Tablet or Capsule Core:

| | Formula | |
|---|---|---|
| Material | Amount (mg/tab) | w/w (%) |
| Tamarind [Tamarindus indica] Seed Extract & Turmeric | 250.0 | 92.59 |
| [Curcuma longa] Rhizome Extract¹ | | |
| Cucumber Standardized Extract[Cucumis sativus]² | 20.0 | 7.41 |
| TOTAL | 270.0 | 100.0 |

¹Commercially available under the tradename Tamaflex ® from Laila Nutraceuticals
²Commercially available under the tradename Q-Actin ® from Iminotech, Inc.

### Example 2

TABLE 2
Active Ingredients for Tablet or Capsule Core including Overages:

| | Formula | |
|---|---|---|
| Material | Amount (mg/tab) | w/w (%) |
| Tamarind [Tamarindus indica] Seed Extract & Turmeric | 262.50ª | 91.62 |
| [Curcuma longa] Rhizome Extract¹ | | |
| Cucumber Standardized Extract[Cucumis sativus]² | 24.0^{b} | 8.38 |
| TOTAL | 286.5 | 100.0 |

¹Commercially available under the tradename Tamaflex ® from Laila Nutraceuticals
²Commercially available under the tradename Q-Actin ® from Iminotech, Inc.
^{a}Overage added to be equal to a 250 mg amount of Tamaflex ®
^{b}Overage added to be equal to a 20 mg amount of Q-Actin ®

### Example 3—Tablet Excipients

In one example, the active ingredients for the tablet core from Examples 1 and 2 may be combined with one or more of the following excipients: dextrose, maltodextrin, croscarmellose, stearic acid, magnesium stearate, pectin, gum acacia, silicon dioxide and polyethylene glycol (PEG).

In an alternative example, the active ingredients for the tablet core from Examples 1 and 2 may be combined with one or more of the following excipients: magnesium stearate, modified starch, powdered cellulose, tricalcium citrate, pregelatinized starch and sodium starch glycolate. See, for example, the various examples below.

In an example, the active ingredients for the tablet core from Examples 1 and 2 may be combined with one or more of the following excipients: carnauba wax, colloidal silicon dioxide, corn starch, hypromellose, iron oxide, magnesium stearate, polydextrose, polyethylene glycol, pregelatinized starch, propylene glycol, shellac, stearic acid, titanium dioxide; or colloidal silicon dioxide, corn starch, croscarmellose sodium, FD&C red no. 40 aluminum lake, iron oxides, microcrystalline cellulose, polyethylene glycol, polyvinyl alcohol, stearic acid, talc, titanium dioxide.

In another example, the active ingredients for the tablet core from Examples 1 and 2 may be combined with one or more of the following excipients: colloidal silicone dioxide, croscarmellose sodium, hypromellose, lactose monohydrate, dextrose, magnesium stearate, microcrystalline cellulose, polyethylene glycol (PEG) and titanium dioxide.

In another example, the active ingredients for the tablet core from Examples 1 and 2 may be combined with one or more of the following excipients: acetylated monoglycerides, colloidal silicon dioxide, corn starch, croscarmellose sodium, methylparaben, microcrystalline cellulose, povidone, pregelatinized starch, propylparaben, sodium benzoate, sodium lauryl sulfate, stearic acid, sucrose, synthetic iron oxide, titanium dioxide, and white wax.

In another example, the active ingredients for the tablet core from Examples 1 and 2 may be combined with one or more of the following excipients: hypromellose, magnesium stearate, microcrystalline cellulose, sodium starch glycolate, croscarmellose sodium, polyethylene glycol, povidone, talc and titanium dioxide.

In another example, the active ingredients for the tablet core from Examples 1 and 2 may be combined with one or more of the following excipients: benzoic acid, carnauba wax, hydroxypropylcellulose, hypromellose, light mineral oil, microcrystalline cellulose, polysorbate 20, povidone, propylene glycol, simethicone emulsion, sorbitan monolaurate, stearic acid, titanium dioxide.

### Example 4—Compressed Tablet

TABLE 3
Formula for Tablet or Caplet Core:

| | | Formula | |
|---|---|---|---|
| | | Amount | |
| Material | Function | (mg/tab) | w/w (%) |
| Tamarind [Tamarindus indica] Seed Extract & | Therapeutic Ingredient | 262.5ª | 78.43 |
| Turmeric [Curcuma longa] Rhizome Extract¹ | | | |
| Cucumber Standardized Extract [Cucumis | Therapeutic Ingredient | 24.0^{b} | 7.17 |
| sativus]² | | | |
| Microcrystalline Cellulose³ | Compression Filler | 40.0 | 11.95 |
| Hydroxypropylcellulose⁴ | Compression Aid | 3.3 | 0.99 |
| Sodium Starch Glycolate⁵ | Disintegrant | 3.3 | 0.99 |
| Magnesium Stearate | Lubricant | 1.6 | 0.48 |
| TOTAL | | 334.7 | 100.0 |

¹Commercially available under the tradename Tamaflex ® from Laila Nutraceuticals
²Commercially available under the tradename Q-Actin ® from Iminotech, Inc.
³Commercially available under the tradename Avicel ® PH101 from the FMC Corporation
⁴Commercially available under the tradename Klucel ™ EXF Ultra HPC from the Ashland Corporation
⁵Commercially available under the tradename Explotab ® from the JRS Corporation
^{a}Overage added to be equal to a 250 mg amount of Tamaflex ®
^{b}Overage added to be equal to a 20 mg amount of Q-Actin ®

### Blending and Compression Steps:

1. The Tamarind Seed Extract and Turmeric Rhizome Extract, as well as the Cucumber extract are added to a suitable V-blender.
2. The microcrystalline cellulose, hydroxypropylcellulose and sodium starch glycolate are also added to the V-blender.
3. The materials from Steps 1 and 2 are blended for 5 minutes.
4. The magnesium stearate is added to the blend from Step 3 and blended for an additional 2 minutes.
5. The blend is transferred to a compression machine and compressed into caplets at not less than 10 kiloNewtons.

### Example 5—Compressed Tablet

TABLE 4
Formula for Tablet or Caplet Core:

| | | Formula | |
|---|---|---|---|
| | | Amount | |
| Material | Function | (mg/tab) | w/w (%) |
| Tamarind [Tamarindus indica] Seed Extract & | Therapeutic Ingredient | 262.5ª | 66.93 |
| Turmeric [Curcuma longa] Rhizome Extract¹ | | | |
| Cucumber Standardized Extract [Cucumis sativus]² | Therapeutic Ingredient | 24.0^{b} | 6.12 |
| Dextrose | Compression Filler | 100.0 | 25.50 |
| Croscarmellose sodium³ | Disintegrant | 3.8 | 0.97 |
| Magnesium Stearate | Lubricant | 1.9 | 0.48 |
| TOTAL | | 392.2 | 100.0 |

¹Commercially available under the tradename Tamaflex ® from Laila Nutraceuticals
²Commercially available under the tradename Q-Actin ® from Iminotech, Inc.
³Commercially available under the tradename Solutab ® from the Roquette Corporation
^{a}Overage added to be equal to a 250 mg amount of Tamaflex ®
^{b}Overage added to be equal to a 20 mg amount of Q-Actin ®

### Blending and Compression Steps:

1. The Tamarind Seed Extract and Turmeric Rhizome Extract, as well as the Cucumber Extract are added to a suitable V-blender.
2. The dextrose and croscarmellose sodium are also added to the V-blender.
3. The materials from Steps 1 and 2 are blended for 5 minutes.
4. The magnesium stearate is added to the blend from Step 3 and blended for an additional 2 minutes.
5. The blend is transferred to a compression machine and compressed into caplets at not less than 10 kiloNewtons.

### Example 6—Capsule Formulation

TABLE 5
Formula for Capsule Fill:

| | | Formula | |
|---|---|---|---|
| | | Amount | |
| Material | Function | (mg/capsule) | w/w (%) |
| Tamarind [Tamarindus indica] Seed Extract | Therapeutic Ingredient | 262.5 | 82.16 |
| & Turmeric [Curcuma longa] Rhizome | | | |
| Extract¹ | | | |
| Cucumber Standardized Extract [Cucumis | Therapeutic Ingredient | 24.0 | 7.51 |
| sativus]² | | | |
| Microcrystalline Cellulose³ | Filler and Tamping | 31.5 | 9.86 |
| | Aid | | |
| Magnesium Stearate | Lubricant | 1.5 | 0.47 |
| TOTAL | | 319.5 | 100.0 |

¹Commercially available under the tradename Tamaflex ® from Laila Nutraceuticals
²Commercially available under the tradename Q-Actin ® from Iminotech, Inc.
³Commercially available under the tradename Avicel ® PH101 from the FMC Corporation
^{a}Overage added to be equal to a 250 mg amount of Tamaflex ®
^{b}Overage added to be equal to a 20 mg amount of Q-Actin ®

### Blending, Tamping and Sealing Steps:

1. The Tamarind Seed Extract and Turmeric Rhizome Extract, as well as the Cucumber Extract are added to a suitable V-blender.
2. The microcrystalline cellulose is also added to the V-blender.
3. The materials from Steps 1 and 2 are blended for 5 minutes.
4. The magnesium stearate is added and blended for an additional 2 minutes.
5. The final blend is added to a capsule filling apparatus wherein hard-shell gelatin capsules are loaded. The blend is dosed into the capsules, tamped and sealed.

### Example 7—Comparative Tablet Formulation

TABLE 6
Comparative Tablet Formulation:

| | | Formula | |
|---|---|---|---|
| | | Amount | |
| Material | Function | (mg/tablet) | w/w (%) |
| Tamarind [Tamarindus indica] Seed Extract | Therapeutic | 262.50ª | 44.16 |
| & Turmeric [Curcuma longa] Rhizome | Ingredient | | |
| Extract¹ | | | |
| Cucumber Standardized Extract [Cucumis | Therapeutic | 24.0^{b} | 4.07 |
| sativus]² | Ingredient | | |
| Microcrystalline Cellulose³ | Compression Aid | 258.77 | 43.53 |
| Crosscarmellose Sodium USP | Disintegrant | 24.23 | 4.08 |
| Silicon Dioxide FCC | Glidant | 3.50 | 0.59 |
| Magnesium Stearate | Lubricant | 15.00 | 2.52 |
| Polyvinyl Alcohol Based Coating⁴ | Coating | 6.47 | 1.09 |
| Purified Water (removed upon processing) | Processing Aid | — | |
| TOTAL | | 594.47 | 100.0 |

¹Commercially available under the tradename Tamaflex ® from Laila Nutraceuticals
²Commercially available under the tradename Q-Actin ® from Iminotech, Inc.
³Commercially available under the tradename Avicel ® PH101 from the FMC Corporation
⁴Coating material containing polyvinyl alcohol, talc, polyethylene glycol, polysorbate 80, commercially available from Colocon Corporation as Opadry ® II
^{a}Overage added to be equal to a 250 mg amount of Tamaflex ®
^{b}Overage added to be equal to a 20 mg amount of Q-Actin ®

### Blending, Tablet Compression and Tablet Coating Steps:

1. The Tamarind Seed Extract and Turmeric Rhizome Extract, and the Cucumber Extract were added to a suitable V-blender.
2. The microcrystalline cellulose, croscarmellose sodium and silicon dioxide were also added to the V-blender.
3. The materials from Steps 1 and 2 were blended for 5 minutes.
4. The magnesium stearate was added and blended for an additional 2 minutes.
5. The final blend was compressed into tablet cores with a target weight of 588.0 mg.
6. The Polyvinyl alcohol based coating was dissolved in purified water while mixing at room temperature.
7. The tablet cores were added to a tablet coating apparatus and coated with the coating material from Step 6 and dried.

### Example 8—Tablet Formulation

TABLE 7
Tablet Formulation:

| | | Formula | |
|---|---|---|---|
| | | Amount | |
| Material | Function | (mg/tablet) | w/w (%) |
| Tamarind [Tamarindus indica] Seed Extract | Therapeutic ingredient | 262.50ª | 39.89 |
| & Turmeric [Curcuma longa] Rhizome | | | |
| Extract¹ | | | |
| Cucumber Standardized Extract [Cucumis | Therapeutic ingredient | 24.0^{b} | 3.65 |
| sativus]² | | | |
| Microcrystalline Cellulose³ | Compression Aid | 50.00 | 7.60 |
| Tricalcium Citrate⁴ | Compression Aid | 258.77 | 39.32 |
| Croscarmellose Sodium USP | Disintegrant | 44.23 | 6.72 |
| Silicon Dioxide FCC | Glidant | 3.50 | 0.53 |
| Magnesium Stearate | Lubricant | 7.00 | 1.06 |
| Polyvinyl Alcohol Based Coating⁵ | Coating | 8.13 | 1.24 |
| Purified Water (removed upon processing) | Processing Aid | — | |
| TOTAL | | 658.13 | 100.0 |

¹Commercially available under the tradename Tamaflex ® from Laila Nutraceuticals
²Commercially available under the tradename Q-Actin ® from Iminotech, Inc.
³Commercially available under the tradename Avicel ® PH102 from the FMC Corporation
⁴Commercially available from the Stauber Corporation
⁵Coating material containing polyvinyl alcohol, talc, polyethylene glycol, polysorbate 80, commercially available from Colorcon Corporation as Opadry ® II
^{a}Overage added to be equal to a 250 mg amount of Tamaflex ®
^{b}Overage added to be equal to a 20 mg amount of Q-Actin ®

### Blending, Tablet Compression and Tablet Coating Steps:

1. The Tamarind Seed Extract and Turmeric Rhizome Extract, and the Cucumber Extract were added to a suitable V-blender.
2. The microcrystalline cellulose, tricalcium citrate, croscarmellose sodium and silicon dioxide were also added to the V-blender.
3. The materials from Steps 1 and 2 were blended for 5 minutes.
4. The magnesium stearate was added and blended for an additional 2 minutes.
5. The final blend was compressed into tablet cores with a target weight of 588.0 mg.
6. The Polyvinyl alcohol based coating was dissolved in purified water while mixing at room temperature.
7. The tablet cores were added to a tablet coating apparatus and coated with the coating material from Step 6 and dried.

### Example 9—Tablet Compression Results

The tablet formulations of Examples 7 and 8 were prepared and subjected to compression and testing, including hardness and friability testing. The results are provided in Tables 8 and 9 below. The tablets in Example 7 were compressed at the following compression forces on a rotary compression machine: 10, 20, 30, 40, 50 and 60 kiloNewtons (kN) at 30-50 RPM in 5 RPM intervals with a final target speed of 50 RPM. The goal was to produce tablets with a hardness of greater than 10 kilopascals (kP). Regardless of the compression force, tablets could not be produced at greater than 10 kP with the formula in Example 7. In addition, these tablets experienced sticking and picking on tablet punch and die tooling surfaces over time, indicating a formula that could not achieve reproducible compression characteristics. Sticking and picking can also lead to loss of portions of tablets on tooling surfaces and uneven tablet weight variation.

The formulation was changed as indicated in Example 8. At a compression force of greater than 20 kN, the desired hardness of greater than 10 kP could be achieved.

TABLE 8
Tablet Compression Results:

| Example | Compression | | Compression | | |
|---|---|---|---|---|---|
| No. | Goal | Pass/Fail | Force (KN) | Hardness (kP) | Notes |
| 7 | Hardness greater | Fail | 35 to 67 | 8-9 kP | This formula |
| | than 10 kp | | | | experienced |
| | without sticking | | | | sticking and |
| | | | | | picking on the |
| | | | | | tablet die and punch |
| | | | | | surfaces |
| 8 | | Pass | 40 | 11.2-12.5 kP | Hardness and |
| | | | | | sticking goals |
| | | | | | achieved |

As shown in Table 9, at a compression force of greater than 20 kN, the desired hardness of greater than 10 kP could be achieved for the revised formula of Example 8 and advantageously maintained at 24 hours after initial compression.

TABLE 9
Tablet Compression Results:

| | Comparative Tablet Formulation | | | |
|---|---|---|---|---|
| | (Example 7) | | | |
| | Immediate | | Tablet Formulation | |
| | Hardness | | (Example 8) | |
| | after | | Initial | 24 Hour |
| Compression | compression | 24 Hour | Hardness | Hardness |
| Force (kN) | (kP) | Hardness (kP) | (kP) | (kP) |
| 10 | 7.1-7.9 | — | 7.0-7.6 | 5.4-6.4 |
| 20 | 10.7-11.1 | — | 14.2-15.8 | 11.7-12.5 |
| 30 | 11.0-11.6 | — | 19.0-20.6 | 13.7-14.9 |
| 40 | 11.2-12.5 | — | 18.9-19.9 | 11.1-14.3 |
| 50 | 10.5-11.4 | — | 18.5-20.1 | 12.5-14.3 |

Hardness 12.0-17.9 kp; Compression Force = 40 +/− 5 kN

### Example 10—Pain Relief Benefits

The compositions described in the Examples above may provide synergistic combinations of active ingredients to deliver enhanced pain relief to customers. For example, the compositions may deliver one or more of the following: 1) improved muscle and joint comfort in just seven (7) days; 2) supports a healthy inflammation response for occasional overworked muscles and stressed joints; 3) enhances occasional stress joints occurring as part of the aging process; 4) reduces aches and pains; 5) works three times faster than glucosamine and chondroitin; 6) reduces muscle soreness; and/or 7) wakes up tired muscles and joints.

### Example 11—Other Consumer Benefits

The compositions described in the Examples above may provide additional benefits to the consumer, for example a smaller size, a smaller dose, or ease of swallowability.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A composition to alleviate pain comprising a combination of Tamarindus indicia, Curcuma longa and Cucumis sativus.

2. The composition of claim 1, wherein the Tamarindus indicia is Tamarindus indicia seed extract.

3. The composition of claim 2, wherein the Curcuma longa is Curcuma longa rhizome extract.

4. The composition of claim 3, wherein the Tamarindus indicia seed extract and the Curcuma longa rhizome extract are used in a ratio ranging from about 1:3 to about 3:1, or about 2:1.

5. The composition of claim 1, wherein the Cucumis sativus is in an amount of about 20 mg, or about 24 mg.

6. The composition of claim 1, wherein the Tamarindus indicia is in an amount of about 157.5 mg, the Curcuma longa is in an amount of about 78.75 mg and the Cucumis sativus is in an amount of about 24 mg.

7. The composition of claim 1, wherein the Tamarindus indicia, Curcuma longa and Cucumis sativus are in a ratio of about 150:75:20.

8. The composition of claim 1, wherein the composition is a pain supplement in a form of a tablet, caplet or hard-shell capsule.

9. The composition of claim 1, wherein the composition is in the form of a compressed tablet.

10. The composition of claim 9, wherein the composition further comprises a compression aid.

11. The composition of claim 10, wherein the compression aid consists of a combination of microcrystalline cellulose and tricalcium citrate.

12. The composition of claim 11, wherein the microcrystalline cellulose is in an amount of about 50 mg and the tricalcium citrate is in an amount of about 258.77 mg.

13. The composition of claim 9, wherein the composition has a hardness of at least 10 kP, or from about 11.2 kP to about 12.5 kP.

14. The composition of claim 1, wherein the composition is free of any active pharmaceutical ingredient (API).

15. The composition of claim 1, wherein the composition is free of dyes.

16. The composition of claim 1, wherein the composition is vegetarian, vegan and/or gluten-free.
